(19) United States
(12) Patent Application Publication
(10) Pub. No.: US 2025/0161550 A1
(43) Pub. Date: May 22, 2025

(54) USING A PIEZO ELECTRIC PUMP TO DETECT WHEN A DRESSING IS FULL AND PREVENT FLUID FROM ENTERING TUBING LINE

(71) Applicant: Solventum Intellectual Properties Company, Maplewood, MN (US)

(72) Inventors: Shannon C. INGRAM, Bulverde, TX (US); Robert HOWARD, Bracknell (GB); Benjamin A. PRATT, Poole (GB)

(21) Appl. No.: 18/729,088

(22) PCT Filed: Jan. 9, 2023
(86) PCT No.: PCT/IB2023/050168

§ 371 Date: Jul. 15, 2024

Related U.S. Application Data
(60) Provisional application No. 63/304,313, filed on Jan. 28, 2022.

(51) Int. Cl. A61M 1/00

(52) U.S. Cl. CPC A61M 1/78; A61M 1/913; A61M 1/962; A61M 1/982; A61M 2205/0294

(57) ABSTRACT

A system for treating a tissue site with negative pressure includes a dressing, a negative-pressure source, a micropump, and a controller. The dressing is configured to be positioned at the tissue site. The negative-pressure source is configured to supply the negative pressure to the dressing through a fluid pathway. The micropump is configured to be positioned in fluid communication with the fluid pathway. The controller is associated with the micropump and configured to generate an alert signal if a deviation condition is met in an actual operating frequency of the micropump.

### CROSS-REFERENCE TO RELATED APPLICATIONS

This application is a U.S. National Stage Entry of International Application No. PCT/IB2023/050168, filed on Jan. 9, 2023, which claims the benefit of priority to U.S. Provisional Application No. 63/304,313, filed on Jan. 28, 2022, each of which are incorporated herein by reference in their entirety.

### TECHNICAL FIELD

The invention set forth in the appended claims relates generally to tissue treatment systems and more particularly, but without limitation, sensor modules and control systems for detecting a presence of liquids in fluid pathways of a negative-pressure wound therapy system.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

There is also widespread acceptance that cleansing a tissue site can be highly beneficial for new tissue growth. For example, a wound or a cavity can be washed out with a liquid solution for therapeutic purposes. These practices are commonly referred to as “irrigation” and “lavage” respectively. “Instillation” is another practice that generally refers to a process of slowly introducing fluid to a tissue site and leaving the fluid for a prescribed period of time before removing the fluid. For example, instillation of topical treatment solutions over a wound bed can be combined with negative-pressure therapy to further promote wound healing by loosening soluble contaminants in a wound bed and removing infectious material. As a result, soluble bacterial burden can be decreased, contaminants removed, and the wound cleansed.

While the clinical benefits of negative-pressure therapy and/or instillation therapy are widely known, improvements to therapy systems, components, and processes may benefit healthcare providers and patients.

### BRIEF SUMMARY

New and useful systems, apparatuses, and methods for detecting the presence of liquid in the fluid pathways of a negative-pressure therapy system are set forth in the appended claims. Illustrative embodiments are also provided to enable a person skilled in the art to make and use the claimed subject matter.

A system for treating a tissue site with negative pressure is presented. The system may include a dressing configured to be positioned at the tissue site, a negative-pressure source configured to supply the negative pressure to the dressing through a fluid pathway, a micropump configured to be positioned in fluid communication with the fluid pathway, and a controller associated with the micropump and configured to generate an alert signal if a deviation condition is met in an actual operating frequency of the micropump.

In other features, the micropump may be configured to be positioned in fluid communication between the dressing and the negative-pressure source in series such that the negative pressure from the negative-pressure source is supplied to the dressing through the micropump. In other features, the negative-pressure source may be configured to supply the negative pressure to the dressing entirely through the micropump. In other features, the micropump may be configured to exhaust gas to the negative-pressure source and/or to an ambient environment. In other features, an intake of the micropump may be configured to be directly exposed to a liquid in the fluid pathway. In other features, the micropump may be a first micropump, and the system may further include a second micropump in fluid communication with the first micropump and the fluid pathway. In other features, an intake of the first micropump may be configured to be directly exposed to a liquid in the fluid pathway, and an intake of the second micropump may be configured to be isolated from the liquid by a liquid-impermeable membrane.

In other features, the system may include a housing having a first cavity fluidly isolated from a second cavity by a walled portion. The micropump may be positioned in the first cavity. The controller may be positioned in the second cavity. In other features, the second cavity may include a power source and a wireless communications interface. In other features, the first cavity may be configured to be positioned in fluid communication with the fluid pathway. In other features, the housing may include a flanged portion surrounding the first cavity. In other features, the flanged portion carries at least one layer of adhesive configured to couple the first cavity in fluid communication with the fluid pathway. In other features, the at least one layer of adhesive may be a plurality of layers of adhesive separated from each other by a release liner positioned between each layer of the plurality of layers of adhesive. The release liner between each of the layers of adhesive may be configured to be removed to expose another of the layers of adhesive. In other features, the micropump may be configured to be fluidly coupled to the fluid pathway through one or more of a cover of the dressing, an in-line conduit between the dressing and the negative-pressure source, a fluid canister between the negative-pressure source and the dressing, and/or a bridge between the negative-pressure source and the dressing.

In other features, the controller may be configured to determine the deviation condition by monitoring the actual operating frequency of the micropump, comparing the actual operating frequency to a target frequency, and determining the deviation condition in response to the actual operating frequency deviating from the targeting frequency. In other features, the controller may be configured to determine the deviation condition by monitoring the actual operating frequency of the micropump, determining whether the actual operating frequency exceeds an upper frequency threshold, and determining the deviation condition in response to the actual operating frequency exceeding the upper frequency threshold. In other features, the controller may be configured to determine the deviation condition by monitoring the actual operating frequency of the micropump, determining whether the actual operating frequency is less than a lower frequency threshold, and determining the deviation condition in response to the actual operating frequency being less than the lower frequency threshold.

In other features, the controller may be configured to determine the deviation condition by monitoring the actual operating frequency of the micropump, determining whether the actual operating frequency approaches zero, and determining the deviation condition in response to the actual operating frequency approaching zero. In other features, the controller may be configured to determine the deviation condition by monitoring the actual operating frequency of the micropump, determining a difference between the actual operating frequency and a target frequency of the micropump, calculating an absolute value of the difference, determining whether the absolute value of the difference exceeds a frequency difference threshold, and determining the deviation condition in response to the absolute value of the difference exceeding the frequency difference threshold.

A system for providing negative-pressure wound therapy is also presented. The system may include a dressing configured to be fluidly coupled to a negative-pressure source, and a liquid-detection sensor module configured to be fluidly coupled to a fluid pathway between the dressing and the negative-pressure source. The liquid-detection sensor module may include a controller and a micropump operatively associated with the controller. The controller may be configured to monitor an actual operating frequency of the micropump, compare the actual operating frequency to a target frequency, determine whether a deviation condition is met when the actual operating frequency deviates from the target frequency, and generate an alert signal if the deviation condition is met.

In other features, the controller may be further configured to determine whether the actual operating frequency exceeds an upper frequency threshold, and determine that the deviation condition is met if the actual operating frequency exceeds the upper frequency threshold. In other features, the controller may be further configured to determine whether the actual operating frequency is less than a lower frequency threshold, and determine that the deviation condition is met if the actual operating frequency is less than the lower frequency threshold. In other features, the controller may be configured to determine whether the actual operating frequency approaches zero, and determine that the deviation condition is met if the actual operating frequency approaches zero. In other features, the controller may be further configured to determine a difference between the actual operating frequency and the target frequency, calculate an absolute value of the difference, determine whether the absolute value of the difference exceeds a frequency difference threshold, and determine that the deviation condition is met if the absolute value of the difference exceeds the frequency difference threshold.

In other features, the controller may be further configured to send the alert signal to one or both of a user device and a therapy unit. In other features, the therapy unit may be configured to receive the alert signal, and shut off a negative-pressure source of the therapy unit in response to receiving the alert signal. In other features, the therapy unit may be configured to receive the alert signal, and generate a user notification in response to receiving the alert signal. In other features, the system includes a user device operatively coupled to the controller. The controller may be further configured to send the alert signal to the user device. The user device may be configured to receive the alert signal, and generate a user notification in response to receiving the alert signal.

A non-transitory computer-readable medium having executable instructions for generating an alert signal indicative of liquid saturation in a therapy system is presented. The executable instructions may configure a controller to monitor an actual operating frequency of a piezoelectric micropump, compare the actual operating frequency to a target frequency, determine whether a deviation condition is met when the actual operating frequency deviates from the target frequency, and generate an alert signal if the deviation condition is met. In other features, the alert signal shuts off a negative-pressure source associated with the therapy system.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a block diagram of an example embodiment of a therapy system that can provide negative-pressure treatment and instillation treatment in accordance with this specification;

FIG. 2 is a block diagram of an example of the liquid-detection sensor module of the therapy system of FIG. 1;

FIG. 3 is a block diagram of another example of the liquid-detection sensor module of the therapy system of FIG. 1;

FIG. 4 is a flowchart of an example process for generating an alert signal in response to a liquid-detection sensor module detecting liquid;

FIG. 5 is a chart illustrating an exemplary operating frequency of a micropump as a function of time when the micropump is pumping a gas;

FIG. 6 is a chart illustrating an exemplary operating frequency of a micropump as a function of time after the micropump has ingested a liquid;

FIG. 7 is a flowchart of another example process for generating an alert signal in response to a liquid-detection sensor module detecting liquid;

FIG. 8 is an isometric view of an assembled example of a dressing with a liquid-detection sensor module;

FIG. 9A is an isometric view of the bottom side of the liquid-detection sensor module of FIG. 8;

FIG. 9B is an isometric view of the top side of the liquid-detection sensor module of FIG. 8;

FIG. 10 is a cross-sectional view of the example dressing and liquid-detection sensor module of FIG. 8, taken at line 10-10, applied to an example tissue site, and illustrating additional details associated with some examples of the therapy system of FIG. 1;

FIG. 11 is an isometric view of an assembled example of a dressing with another example of a liquid-detection sensor module;

FIG. 12A is an isometric view of the bottom side of the liquid-detection sensor module of FIG. 11;

FIG. 12B is an isometric view of the top side of the liquid-detection sensor module of FIG. 11;

FIG. 13 is a cross-sectional view of an example dressing and liquid-detection sensor module of FIG. 11, taken at line 13-13, applied to an example tissue site, and illustrating additional details associated with some examples of the therapy system of FIG. 1.

FIG. 14 is an isometric view of an assembled example of a dressing with another example of a liquid-detection sensor module;

FIG. 15A is an isometric view of the bottom side of the liquid-detection sensor module of FIG. 14.

FIG. 15B is an isometric view of the top side of the liquid-detection sensor module of FIG. 14.

FIG. 16 is a cross-sectional view of the example dressing and liquid-detection sensor module of FIG. 14, taken at line 16-16, applied to an example tissue site, and illustrating additional details associated with some examples of the therapy system of FIG. 1.

FIG. 17 is an isometric view of an assembled example of a dressing with a liquid-detection sensor module fluidly coupled to the dressing through a conduit and a bridge; and

FIG. 18 is a cross-sectional view of a container with a liquid-detection sensor module disposed in an interior space of the container.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

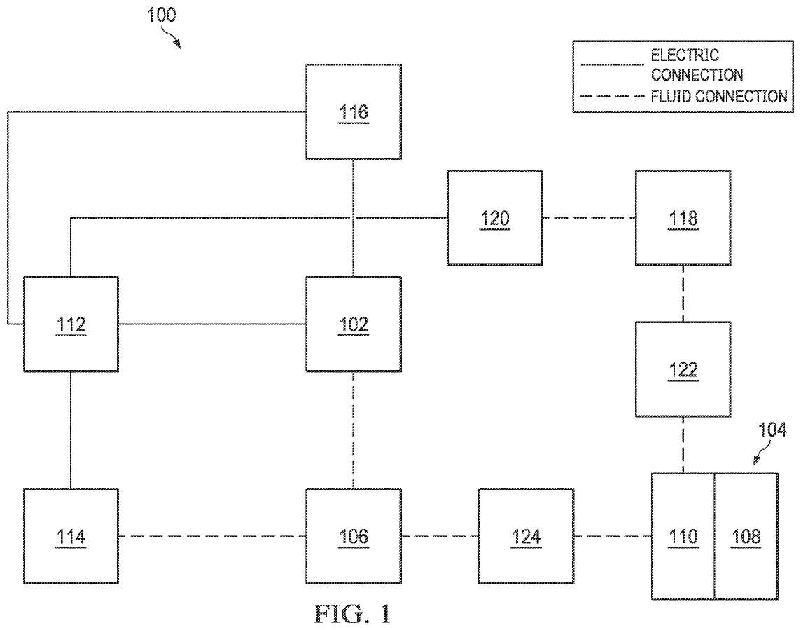

FIG. 1 is a block diagram of an example embodiment of a therapy system 100 that can provide negative-pressure therapy with instillation of topical treatment solutions to a tissue site in accordance with this specification.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The therapy system 100 may include a source or supply of negative pressure, such as a negative-pressure source 102, and one or more distribution components. A distribution component is preferably detachable and may be disposable, reusable, or recyclable. A dressing, such as a dressing 104, and a fluid container, such as a container 106, are examples of distribution components that may be associated with some examples of the therapy system 100. As illustrated in the example of FIG. 1, the dressing 104 may comprise or consist essentially of a tissue interface 108, a cover 110, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 104. For example, such a dressing interface may be a SENSAT.R.A.C.™ Pad available from Kinetic Concepts, Inc. of San Antonio, Texas.

The therapy system 100 may also include a regulator or controller, such as a controller 112. Additionally, the therapy system 100 may include sensors to measure operating parameters and provide feedback signals to the controller 112 indicative of the operating parameters. As illustrated in FIG. 1, for example, the therapy system 100 may include a first sensor 114 and a second sensor 116 coupled to the controller 112.

The therapy system 100 may also include a source of instillation solution. For example, a solution source 118 may be fluidly coupled to the dressing 104, as illustrated in the example embodiment of FIG. 1. The solution source 118 may be fluidly coupled to a positive-pressure source such as a positive-pressure source 120, a negative-pressure source such as the negative-pressure source 102, or both in some embodiments. A regulator, such as an instillation regulator 122, may also be fluidly coupled to the solution source 118 and the dressing 104 to ensure proper dosage of instillation solution (e.g. saline) to a tissue site. For example, the instillation regulator 122 may comprise a piston that can be pneumatically actuated by the negative-pressure source 102 to draw instillation solution from the solution source during a negative-pressure interval and to instill the solution to a dressing during a venting interval. Additionally or alternatively, the controller 112 may be coupled to the negative-pressure source 102, the positive-pressure source 120, or both, to control dosage of instillation solution to a tissue site. In some embodiments, the instillation regulator 122 may also be fluidly coupled to the negative-pressure source 102 through the dressing 104, as illustrated in the example of FIG. 1.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 102 may be combined with the controller 112, the solution source 118, and other components into a therapy unit.

In general, components of the therapy system 100 may be coupled directly or indirectly. For example, the negative-pressure source 102 may be directly coupled to the container 106 and may be indirectly coupled to the dressing 104 through the container 106. Coupling may include fluid, mechanical, thermal, electrical, or chemical coupling (such as a chemical bond), or some combination of coupling in some contexts. For example, the negative-pressure source 102 may be electrically coupled to the controller 112 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 102, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 102 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 106 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 112, may be a microprocessor or computer programmed to operate one or more components of the therapy system 100, such as the negative-pressure source 102. In some embodiments, for example, the controller 112 may be a microcontroller, which generally comprises an integrated circuit containing a processor core and a memory programmed to directly or indirectly control one or more operating parameters of the therapy system 100. Operating parameters may include the power applied to the negative-pressure source 102, the pressure generated by the negative-pressure source 102, or the pressure distributed to the tissue interface 108, for example. The controller 112 is also preferably configured to receive one or more input signals, such as a feedback signal, and programmed to modify one or more operating parameters based on the input signals.

Sensors, such as the first sensor 114 and the second sensor 116, may be any apparatus operable to detect or measure a physical phenomenon or property, and generally provide a signal indicative of the phenomenon or property that is detected or measured. For example, the first sensor 114 and the second sensor 116 may be configured to measure one or more operating parameters of the therapy system 100. In some embodiments, the first sensor 114 may be a transducer configured to measure pressure in a pneumatic pathway and convert the measurement to a signal indicative of the pressure measured. In some embodiments, for example, the first sensor 114 may be a piezo-resistive strain gauge. The second sensor 116 may optionally measure operating parameters of the negative-pressure source 102, such as a voltage or current, in some embodiments. Preferably, the signals from the first sensor 114 and the second sensor 116 are suitable as an input signal to the controller 112, but some signal conditioning may be appropriate in some embodiments. For example, the signal may need to be filtered or amplified before it can be processed by the controller 112. Typically, the signal is an electrical signal, but may be represented in other forms, such as an optical signal.

The tissue interface 108 can be generally adapted to partially or fully contact a tissue site. The tissue interface 108 may take many forms, and may have many sizes, shapes, or thicknesses, depending on a variety of factors, such as the type of treatment being implemented or the nature and size of a tissue site. For example, the size and shape of the tissue interface 108 may be adapted to the contours of deep and irregular shaped tissue sites. Any or all of the surfaces of the tissue interface 108 may have an uneven, coarse, or jagged profile.

In some embodiments, the tissue interface 108 may comprise or consist essentially of a manifold. A manifold in this context may comprise or consist essentially of a means for collecting or distributing fluid through the tissue interface 108 under pressure. For example, a manifold may be adapted to receive negative pressure from a source and distribute negative pressure through multiple apertures through the tissue interface 108, which may have the effect of collecting fluid from a tissue site and drawing the fluid toward the source. In some embodiments, the fluid path may be reversed or a secondary fluid path may be provided to facilitate delivering fluid, such as fluid from a source of instillation solution, to a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the tissue interface 108 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the tissue interface 108 may also vary according to needs of a prescribed therapy. For example, the tensile strength of foam may be increased for instillation of topical treatment solutions. The 25% compression load deflection of the tissue interface 108 may be at least 0.35 pounds per square inch, and the 65% compression load deflection may be at least 0.43 pounds per square inch. In some embodiments, the tensile strength of the tissue interface 108 may be at least 10 pounds per square inch. The tissue interface 108 may have a tear strength of at least 2.5 pounds per inch. In some embodiments, the tissue interface may be foam comprised of polyols such as polyester or polyether, isocyanate such as toluene diisocyanate, and polymerization modifiers such as amines and tin compounds. In some examples, the tissue interface 108 may be reticulated polyurethane foam such as found in GRANUFOAM™ dressing or V.A.C. VERAFLO™ dressing, both available from Kinetic Concepts, Inc. of San Antonio, Texas.

The thickness of the tissue interface 108 may also vary according to needs of a prescribed therapy. For example, the thickness of the tissue interface may be decreased to reduce tension on peripheral tissue. The thickness of the tissue interface 108 can also affect the conformability of the tissue interface 108. In some embodiments, a thickness in a range of about 5 millimeters to 10 millimeters may be suitable.

The tissue interface 108 may be either hydrophobic or hydrophilic. In an example in which the tissue interface 108 may be hydrophilic, the tissue interface 108 may also wick fluid away from a tissue site, while continuing to distribute negative pressure to the tissue site. The wicking properties of the tissue interface 108 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Texas. Other hydrophilic foams may include those made from polyether. Other foams that may exhibit hydrophilic characteristics include hydrophobic foams that have been treated or coated to provide hydrophilicity.

In some embodiments, the tissue interface 108 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. The tissue interface 108 may further serve as a scaffold for new cell-growth, or a scaffold material may be used in conjunction with the tissue interface 108 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 110 may provide a bacterial barrier and protection from physical trauma. The cover 110 may also be constructed from a material that can reduce evaporative losses and provide a fluid seal between two components or two environments, such as between a therapeutic environment and a local external environment. The cover 110 may comprise or consist of, for example, an elastomeric film or membrane that can provide a seal adequate to maintain a negative pressure at a tissue site for a given negative-pressure source. The cover 110 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 110 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 110 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minnesota; polyurethane (PU) drape, commercially available from Avery Dennison Corporation, Pasadena, California; polyether block polyamide copolymer (PEBAX), for example, from Arkema S. A., Colombes, France; and Inspire 2301 and Inspire 2327 polyurethane films, commercially available from Expopack Advanced Coatings, Wrexham, United Kingdom. In some embodiments, the cover 110 may comprise INSPIRE 2301 having an MVTR (upright cup technique) of 2600 g/m²/24 hours and a thickness of about 30 microns.

An attachment device may be used to attach the cover 110 to an attachment surface, such as undamaged epidermis, a gasket, or another cover. The attachment device may take many forms. For example, an attachment device may be a medically-acceptable, pressure-sensitive adhesive configured to bond the cover 110 to epidermis around a tissue site. In some embodiments, for example, some or all of the cover 110 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

The solution source 118 may also be representative of a container, canister, pouch, bag, or other storage component, which can provide a solution for instillation therapy. Compositions of solutions may vary according to a prescribed therapy, but examples of solutions that may be suitable for some prescriptions include hypochlorite-based solutions, silver nitrate (0.5%), sulfur-based solutions, biguanides, cationic solutions, and isotonic solutions.

In operation, the tissue interface 108 may be placed within, over, on, or otherwise proximate to a tissue site. If the tissue site is a wound, for example, the tissue interface 108 may partially or completely fill the wound, or it may be placed over the wound. The cover 110 may be placed over the tissue interface 108 and sealed to an attachment surface near a tissue site. For example, the cover 110 may be sealed to undamaged epidermis peripheral to a tissue site. Thus, the dressing 104 can provide a sealed therapeutic environment proximate to a tissue site, substantially isolated from the external environment, and the negative-pressure source 102 can reduce pressure in the sealed therapeutic environment.

The process of reducing pressure may be described illustratively herein as “delivering,” “distributing,” or “generating” negative pressure, for example.

In general, exudate and other fluid flow toward lower pressure along a fluid path. Thus, the term “downstream” typically implies a position in a fluid path relatively closer to a source of negative pressure or further away from a source of positive pressure. Conversely, the term “upstream” implies a position relatively further away from a source of negative pressure or closer to a source of positive pressure.

Negative pressure applied to the tissue site through the tissue interface 108 in the sealed therapeutic environment can induce macro-strain and micro-strain in the tissue site. Negative pressure can also remove exudate and other fluid from a tissue site, which can be collected in container 106.

In some embodiments, the controller 112 may receive and process data from one or more sensors, such as the first sensor 114. The controller 112 may also control the operation of one or more components of the therapy system 100 to manage the pressure delivered to the tissue interface 108. In some embodiments, controller 112 may include an input for receiving a desired target pressure and may be programmed for processing data relating to the setting and inputting of the target pressure to be applied to the tissue interface 108. In some example embodiments, the target pressure may be a fixed pressure value set by an operator as the target negative pressure desired for therapy at a tissue site and then provided as input to the controller 112. The target pressure may vary from tissue site to tissue site based on the type of tissue forming a tissue site, the type of injury or wound (if any), the medical condition of the patient, and the preference of the attending physician. After selecting a desired target pressure, the controller 112 can operate the negative-pressure source 102 in one or more control modes based on the target pressure and may receive feedback from one or more sensors to maintain the target pressure at the tissue interface 108.

In some embodiments, the controller 112 may have a continuous pressure mode, in which the negative-pressure source 102 is operated to provide a constant target negative pressure for the duration of treatment or until manually deactivated. Additionally or alternatively, the controller may have an intermittent pressure mode. In example embodiments, the controller 112 can operate the negative-pressure source 102 to cycle between a target pressure and atmospheric pressure. For example, the target pressure may be set at a value of 135 mmHg for a specified period of time (e.g., five minutes), followed by a specified period of time (e.g., two minutes) of deactivation. The cycle can be repeated by activating the negative-pressure source 102, which can form a square wave pattern between the target pressure and atmospheric pressure.

In some example embodiments, the increase in negative-pressure from ambient pressure to the target pressure may not be instantaneous. For example, the negative-pressure source 102 and the dressing 104 may have an initial rise time. The initial rise time may vary depending on the type of dressing and therapy equipment being used. For example, the initial rise time for one therapy system may be in a range of about 20-30 mmHg/second and in a range of about 5-10 mmHg/second for another therapy system. If the therapy system 100 is operating in an intermittent mode, the repeating rise time may be a value substantially equal to the initial rise time.

In some example dynamic pressure control modes, the target pressure can vary with time. For example, the target pressure may vary in the form of a triangular waveform, varying between a negative pressure of 50 and 135 mmHg with a rise rate of negative pressure set at a rate of 25 mmHg/min. and a descent rate set at 25 mmHg/min. In other embodiments of the therapy system 100, the triangular waveform may vary between negative pressure of 25 and 135 mmHg with a rise rate of about 30 mmHg/min. and a descent rate set at about 30 mmHg/min.

In some embodiments, the controller 112 may control or determine a variable target pressure in a dynamic pressure mode, and the variable target pressure may vary between a maximum and minimum pressure value that may be set as an input prescribed by an operator as the range of desired negative pressure. The variable target pressure may also be processed and controlled by the controller 112, which can vary the target pressure according to a predetermined waveform, such as a triangular waveform, a sine waveform, or a saw-tooth waveform. In some embodiments, the waveform may be set by an operator as the predetermined or time-varying negative pressure desired for therapy.

In some embodiments, the controller 112 may receive and process data, such as data related to instillation solution provided to the tissue interface 108. Such data may include the type of instillation solution prescribed by a clinician, the volume of fluid or solution to be instilled to a tissue site (“fill volume”), and the amount of time prescribed for leaving solution at a tissue site (“dwell time”) before applying a negative pressure to the tissue site. The fill volume may be, for example, between 10 and 500 mL, and the dwell time may be between one second to 30 minutes. The controller 112 may also control the operation of one or more components of the therapy system 100 to instill solution. For example, the controller 112 may manage fluid distributed from the solution source 118 to the tissue interface 108. In some embodiments, fluid may be instilled to a tissue site by applying a negative pressure from the negative-pressure source 102 to reduce the pressure at the tissue site, drawing solution into the tissue interface 108. In some embodiments, solution may be instilled to a tissue site by applying a positive pressure from the positive-pressure source to move solution from the solution source 118 to the tissue interface 108. Additionally or alternatively, the solution source 118 may be elevated to a height sufficient to allow gravity to move solution into the tissue interface 108.

The controller 112 may also control the fluid dynamics of instillation by providing a continuous flow of solution or an intermittent flow of solution. Negative pressure may be applied to provide either continuous flow or intermittent flow of solution. The application of negative pressure may be implemented to provide a continuous pressure mode of operation to achieve a continuous flow rate of instillation solution through the tissue interface 108, or it may be implemented to provide a dynamic pressure mode of operation to vary the flow rate of instillation solution through the tissue interface 108. Alternatively, the application of negative pressure may be implemented to provide an intermittent mode of operation to allow instillation solution to dwell at the tissue interface 108. In an intermittent mode, a specific fill volume and dwell time may be provided depending, for example, on the type of tissue site being treated and the type of dressing being utilized. After or during instillation of solution, negative-pressure treatment may be applied. The controller 112 may be utilized to select a mode of operation and the duration of the negative pressure treatment before commencing another instillation cycle by instilling more solution.

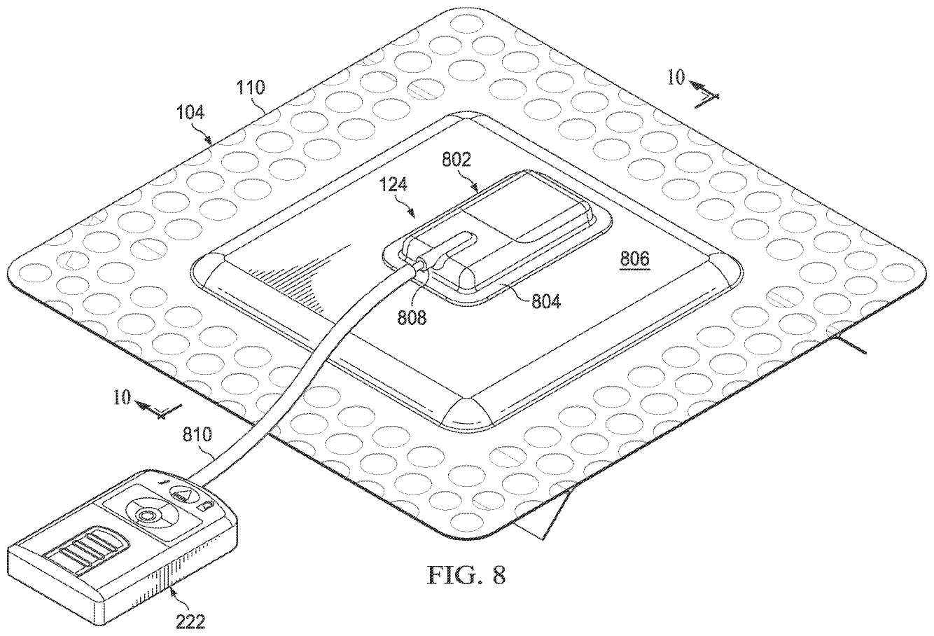

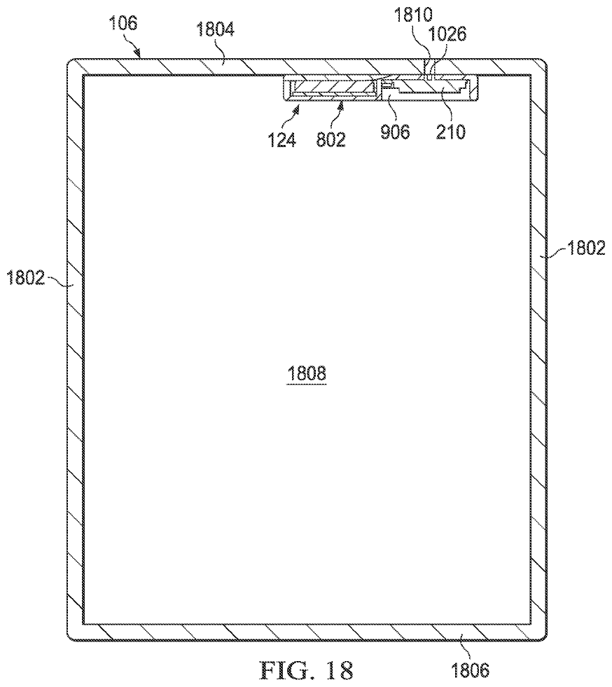

In various implementations, the therapy system 100 include one or more sensors, such as a liquid-detection sensor module 124, for detecting a presence of fluids in the liquid pathways and/or components of the system. For example, the liquid-detection sensor module 124 may be fluidly coupled to one or more of the fluid conductors of the therapy system 100. As shown in the example of FIG. 1, the liquid-detection sensor module 124 may be fluidly coupled in-line or in series between the container 106 and the dressing 104. For example, the liquid-detection sensor module 124 may be fluidly coupled by a first fluid conductor to the dressing 104, and fluidly coupled by a second fluid conductor to the container 106. In various implementations, the liquid-detection sensor module 124 may be directly coupled or attached to one or more components of the dressing 104 as shown, without limitation, in FIGS. 8, 10, 11, and 13. In various implementations, the liquid-detection sensor module 124 may be directly coupled or attached to the container 106 as shown, without limitation, in FIG. 18. For example, the liquid-detection sensor module 124 may be directly coupled or attached to an interior space of the container 106. In various implementations, the liquid-detection sensor module 124 may detect the presence of liquids. For example, if the liquid-detection sensor module 124 is fluidly coupled to a fluid conductor, the liquid-detection sensor module 124 may detect the presence of liquids within the fluid conductor. In various implementations, if the liquid-detection sensor module 124 is coupled or attached to the interior space of the container 106, the liquid-detection sensor module 124 may detect when a level of liquids within the container 106 rises to the level of the liquid-detection sensor module 124.

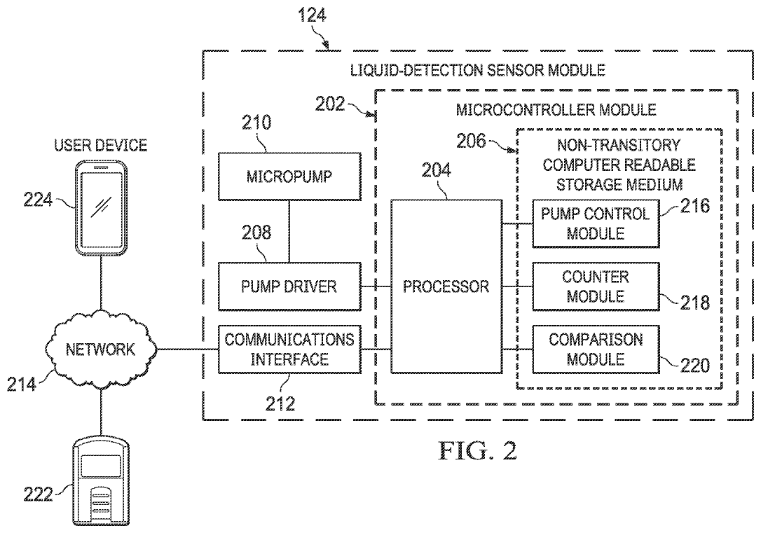

FIG. 2 is a block diagram of an example liquid-detection sensor module 124 suitable for use in the therapy system 100 of FIG. 1. In various implementations, the liquid-detection sensor module 124 may include a microcontroller module 202. The microcontroller module 202 may include a processor 204 operatively coupled to a non-transitory computer readable storage medium 206. In various implementations, the non-transitory computer readable storage medium 206 may include single-level cell (SLC) NAND flash, multi-level cell (MLC) NAND flash, triple-level cell (TLC) NAND flash, quad-level cell (QLC) NAND flash, NOR flash, or any other suitable non-volatile memory or non-volatile storage medium accessible by the processor 204 with the exception of a transitory signal. In various implementations, the liquid-detection sensor module 124 may also include a pump driver 208. The pump driver 208 may be any device capable of generating an electrical signal capable of driving a pump. For example, the pump driver 208 may be capable of generating and outputting a direct current (DC) waveform by varying the output voltage over time. In various implementations, the pump driver 208 may generate and output a sine, square, ramp, triangle, or pulse waveform having a user-selectable frequency and amplitude (voltage). In various implementations, the pump driver 208 may be operatively coupled to and controlled by the processor 204.

In various implementations, the liquid-detection sensor module 124 may include a pump, such as a micropump 210. The micropump 210 may be piezoelectric. In some examples, the micropump 210 may be an air pump, such as a piezoelectric microblower. Piezoelectric implementations of the micropump 210 may have a diaphragm coupled to a piezoelectric element. As an electrical current is applied to the piezoelectric element, the piezoelectric element deforms, causing a corresponding deformation of the diaphragm. As the diaphragm deforms, the micropump 210 draws air into an inlet or pushes air out from an outlet of a housing of the micropump 210. The micropump 210 may be operatively coupled to and driven by the pump driver 208. For example, if the pump driver 208 outputs a square wave to the piezoelectric element of the micropump 210, then the piezoelectric element may have a first shape when the output from the pump driver 208 is at zero volts, and a second shape when the output from the pump driver 208 is at the target voltage. Thus, the oscillation frequency of the diaphragm of the micropump 210 may be correlated to the frequency of the square wave (or other waveform) output by the pump driver 208 (e.g., the target voltage). In various implementations, the Microblower MZB3004T04 piezoelectric air pump available from Murata Manufacturing Co., Ltd. may be used as the micropump 210.

In various implementations, the liquid-detection sensor module 124 may include a communications interface 212. The communications interface 212 may be a communications device, such as a transceiver, suitable for sending and receiving data wirelessly or via a wired connection over a network 214. In various implementations, the network 214 may be a local area network (LAN) or a wireless personal area network (WPAN). Examples of a LAN include Institute of Electrical and Electronics Engineers (IEEE) Standard 802.11-2020 (also known as the WIFI wireless networking standard) and IEEE Standard 802.3-2015 (also known as the ETHERNET wired networking standard). Examples of a WPAN include IEEE Standard 802.15.4 (including the ZIGBEE standard from the ZigBee Alliance) and, from the Bluetooth Special Interest Group (SIG), the BLUETOOTH wireless networking standard (including Core Specification versions 3.0, 4.0, 4.1, 4.2, 5.0, and 5.1 from the Bluetooth SIG). In various implementations, the liquid-detection sensor module 124 may be operatively coupled to the processor 204.

In various implementations, the non-transitory computer readable storage medium 206 may include one or more software modules, such as a pump control module 216, a counter module 218, and/or a comparison module 220. In various implementations, the pump control module 216, the counter module 218, and the comparison module 220 may each contain instructions capable of being executed by the processor 204 to perform various functions. For example, the pump control module 216 may contain instructions that configure the processor 204 to control the pump driver 208 based on a target frequency a target voltage. Both the target frequency and the target voltage may, without limitation, be selected by a user or input as a pre-programmed setting. In various implementations, the counter module 218 may contain instructions that configure the processor 204 to measure the actual operating frequency of the micropump 210. For example, the actual operating frequency of the micropump 210 may be the frequency with which the piezoelectric element and/or the diaphragm of the micropump 210 deforms. In various implementations, the comparison module 220 may contain instructions that configure the processor 204 to compare the measured actual operating frequency against the target frequency. If the difference between the operating frequency and the target frequency exceeds a threshold, then the comparison module 220 may contain instructions for the processor 204 to send an alarm signal. For example, the comparison module 220 may configure the processor 204 to send an alarm signal to a therapy unit 222 or a user device 224 via the communications interface 212 and the network 214. In various implementations, the therapy unit 222 may include at least the negative-pressure source 102 and the controller 112. The alarm signal may cause the controller 112 of the therapy unit 222 to generate an audible, visual, and/or tactile alert, and/or shut off the negative-pressure source 102. Similarly, the user device 224 may also generate an audible, visual, and/or tactile alert to notify the user that the liquid-detection sensor module 124 detects the presence of liquid.

In various implementations, the user device 224 may also include a communication device, and may be a smartphone, a tablet computer, or other device that is capable of storing a software application programmed for a specific operating system (e.g., IOS, Android, and Windows). The user device 224 may also include an electronic display and a graphical user interface (GUI), for providing visual images and messages to a user, such as a clinician or patient. The user device 224 may be configured to communicate with one or more networks, such as network 214. In some embodiments, the user device 224 may include a cellular modem and may be configured to communicate with the network 214 through a cellular connection. In other embodiments, user device 224 may include a BLUETOOTH radio or other wireless radio technology for communicating with the network 214. In various implementations, a near-field communication (NFC) tag may be provided on each dressing 104 and/or liquid-detection sensor module 124. In various implementations, the therapy unit 222 and/or the user device 224 may include an NFC reader for pairing the therapy unit 222 and/or the user device 224 with the liquid-detection sensor module 124.

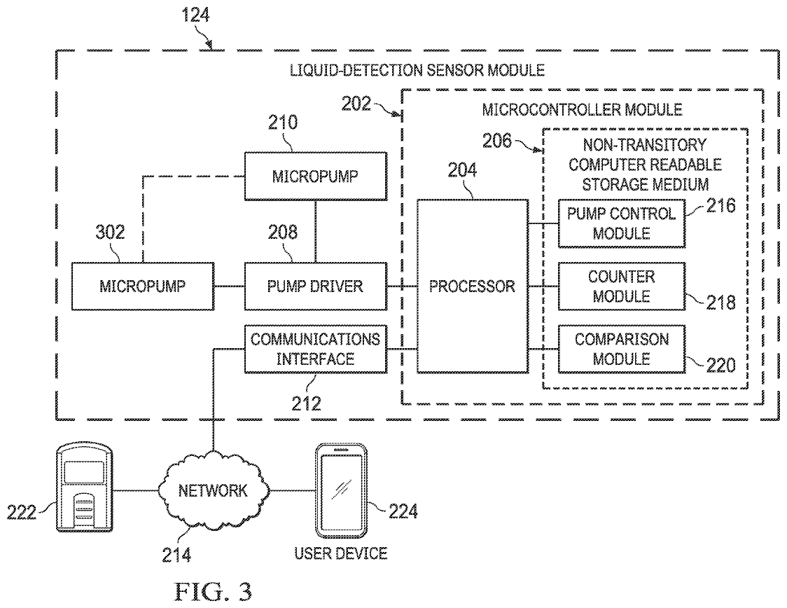

FIG. 3 is a block diagram of another example liquid-detection sensor module 124 suitable for use in the therapy system 100 of FIG. 1. In various implementations, the liquid-detection sensor module 124 may include a second micropump, such as a micropump 302, which may be piezoelectric. The micropump 302 may be operatively coupled to the pump driver 208, and fluidly coupled to the micropump 210. The micropump 302 may be driven and/or controlled by the pump driver 208, which may be in turn controlled by the processor 204. In various implementations, the micropump 302 may provide negative pressure to the micropump 210 in order to draw any liquid that has entered the micropump 210 out from the micropump 210. In various implementations, the micropump 302 may be fluidly coupled to the dressing 104, and be configured to provide negative pressure to the dressing 104. For example, the micropump 302 may be configured to provide negative pressure to the dressing 104 in response to a leak detected in the therapy system 100.

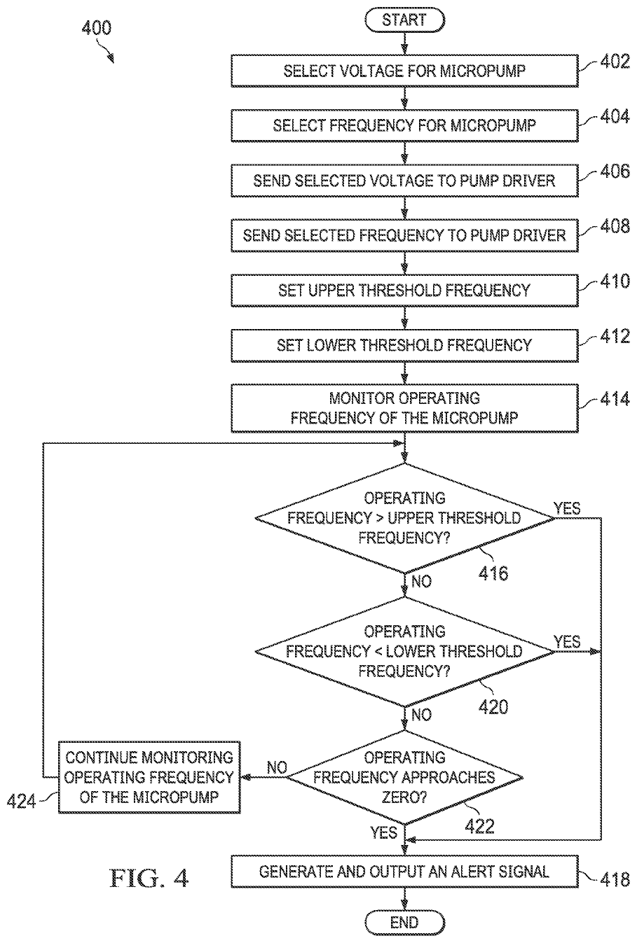

FIG. 4 is a flowchart of an example process 400 which may be performed by the processor 204 and the pump control module 216, counter module 218, and/or the comparison module 220 in order to generate an alert signal in response to determining that liquid has entered the micropump 210. Control, such as with the processor 204, may begin at 402. At 402, the processor 204 may access the pump control module 216 and select a target voltage for operating the micropump 210. Control proceeds to 404. At 404, the processor 204 may access the pump control module and select a target frequency for operating the micropump 210. Control proceeds to 406. At 406, the processor 204 sends the selected target voltage to the pump driver 208. Control proceeds to 408. At 408, the processor 204 sends the selected target frequency to the pump driver 208. After receiving the selected target voltage and the selected target frequency from the processor 204, the pump driver 208 may supply the piezoelectric micropump with a DC waveform, such as a triangular waveform, a sine waveform, or a saw-tooth waveform, having the target voltage and the target frequency. Control proceeds to 410.

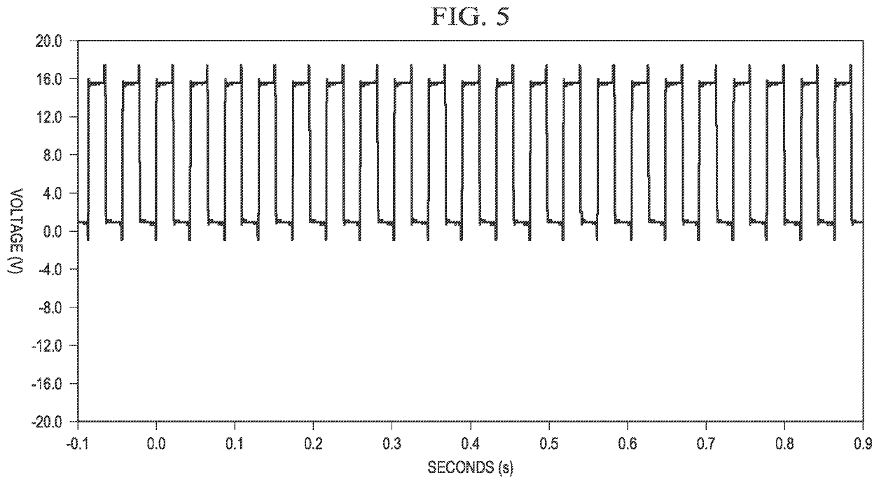

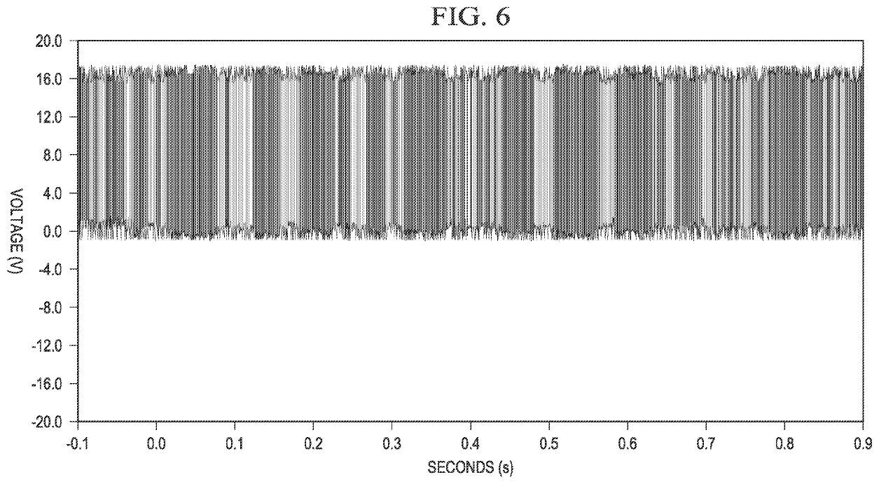

At 410, the processor 204 accesses the pump control module 216 and/or the comparison module 220 in order to determine and/or set an upper threshold frequency based on the selected target frequency. In various implementations, when the micropump 210 is pumping a gas, such as air, the micropump 210 runs at an actual operating frequency that is at or near the selected target frequency output to it by the pump driver 208. Thus, the processor 204 may determine and set the upper threshold frequency at start-up or another time to a value proximate to or based on the target frequency when the micropump 210 is known to be pumping a gas. In other examples, the upper threshold frequency may be set by a user or provided as a pre-programmed setting based, for example, on the manufacturer specification for the particular micropump. FIG. 5 is a chart illustrating an example operating frequency of the micropump 210 as a function of time when the micropump 210 is pumping a gas. However, if a liquid enters the micropump 210, the micropump 210 will run at an actual operating frequency deviating from the selected target frequency. FIG. 6 is a chart illustrating an example operating frequency of the micropump 210 as a function of time after liquid has entered the micropump 210. As can be seen in FIGS. 5 and 6, after liquid enters the micropump 210, the actual operating frequency of the micropump 210 may increase. In other examples, the actual operating frequency of the micropump 210 may decrease or reach zero after liquid enters the micropump 210. Referring back to FIG. 4, at 410, the upper threshold frequency may be set based on the selected target frequency and the characteristics of the micropump 210 such that when liquid enters the micropump 210, the micropump 210 operates at an actual operating frequency at or above the upper threshold frequency. Control proceeds to 412.

At 412, the processor 204 accesses the pump control module 216 and/or the comparison module 220 in order to determine and/or set a lower threshold frequency based on the selected target frequency. The lower threshold frequency may be set based on the selected target frequency and the operating characteristics of the micropump 210 when exposed to a liquid such that when liquid enters the micropump 210, the micropump 210 operates at an actual operating frequency at or below the lower threshold frequency. Control proceeds to 414. At 414, the processor 204 accesses the counter module 218 and monitors the actual operating frequency of the micropump 210. Control proceeds to 416. At 416, the processor 204 accesses the counter module 218 and the comparison module 220 to determine if the actual operating frequency of the micropump 210 exceeds the upper threshold frequency. If at 416, the processor 204 determines that the actual operating frequency of the micropump 210 exceeds the upper threshold frequency set at 410, control proceeds to 418 to generate and output an alert signal. Otherwise, control proceeds to 420.

At 418, the processor 204 accesses the comparison module 220 and generates an alert signal. In various implementations, if the liquid-detection sensor module 124 is directly coupled to the dressing 104 or coupled to in-line conduits between the dressing 104 and the container 106, then the alert signal may indicate that the dressing 104 is saturated with liquid and is full. In various implementations, if the liquid-detection sensor module 124 is coupled or attached to an interior space of the container 106, then the alert signal may indicate that level of liquid collected within the container 106 has reached the level of the liquid-detection sensor module 124. In various implementations, if the liquid-detection sensor module 124 is coupled or attached to the interior space of the container 106 near the top of the container 106, then the alert signal may indicate that the container 106 is full. The processor 204 may send the alert signal to the therapy unit 222 and/or the user device 224 via the communications interface 212 and/or the network 214. In response to receiving the alert signal, the therapy unit 222 and/or the user device 224 may generate and output a visual, audible, and/or tactile signal to indicate to the user that the dressing 104 is saturated and/or the container 106 is full, as appropriate. In various implementations, the controller 112 of the therapy unit 222 may shut off the negative-pressure source 102 of the therapy unit in response to receiving the alert signal.

At 420, the processor 204 accesses the counter module 218 and the comparison module 220 to determine if the actual operating frequency of the micropump 210 is less than the lower threshold frequency set at 412. If the processor 204 determines that the actual operating frequency of the piezoelectric micropump is less than the lower threshold frequency, control proceeds to 418 to generate and output an alert signal. Otherwise, control proceeds to 422. At 422, the processor 204 accesses the counter module 218 and the comparison module 220 to determine if the actual operating frequency of the micropump 210 approaches zero. For example, the actual operating frequency of the micropump 210 may be considered to approach zero if it is within a distance of zero. If at 422, this condition is met, control proceeds to 418 to generate and output and alert signal. Otherwise, control proceeds to 424. At 424, the processor accesses the counter module 218 and continues monitoring the actual operating frequency of the micropump 210. Control proceeds back to 416.

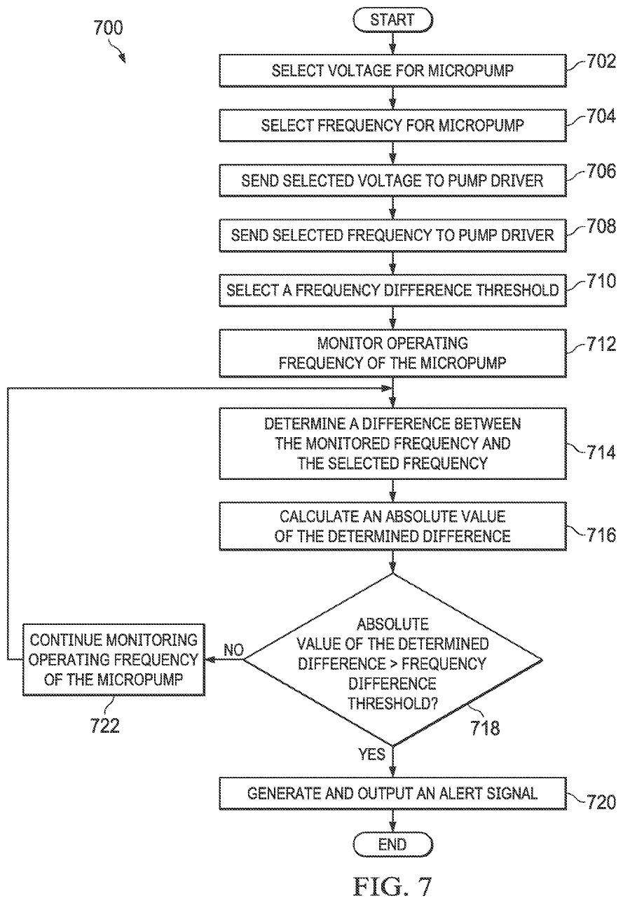

FIG. 7 is a flowchart of an example process 400 which may be performed by the processor 204 and the pump control module 216, counter module 218, and/or the comparison module 220 in order to generate an alert signal in response to determining that liquid has entered the micropump 210. Control, such as with the processor 204, may begin at 702. At 702, the processor 204 may access the pump control module 216 and select a target voltage for operating the micropump 210. Control proceeds to 704. At 704, the processor 204 may access the pump control module and select a target frequency for operating the micropump 210. Control proceeds to 706. At 706, the processor 204 sends the selected target voltage to the pump driver 208. Control proceeds to 708. At 708, the processor 204 sends the selected target frequency to the pump driver 208. After receiving the selected target voltage and the selected target frequency from the processor 204, the pump driver 208 may supply the piezoelectric micropump with a DC waveform, such as a triangular waveform, a sine waveform, or a saw-tooth waveform, having the target voltage and the target frequency. Control proceeds to 710.

At 710, the processor 204 accesses the pump control module 216 and/or the comparison module 220 in order to determine and/or set a frequency difference threshold. As previously described, the actual operating frequency of the micropump 210 may deviate from the selected target frequency after liquid enters the micropump 210. In various implementations, the frequency difference threshold may be selected or set based on a magnitude of a difference between the actual operating frequency and the selected target frequency of the micropump 210 that would indicate that liquid has entered the micropump 210. In some examples, the difference between the actual operating frequency and the selected target frequency may be measured in a bench test as a micropump suitable for use as the micropump 210 is exposed to a liquid. In other examples, the difference between the actual operating frequency and the selected target frequency may be provided as a manufacturer specification. Control proceeds to 712. At 712, the processor 204 accesses the counter module 218 and monitors the actual operating frequency of the micropump 210. Control proceeds to 714.

At 714, the processor 204 accesses the pump control module 216, the counter module 218, and/or the comparison module 220 in order to determine a difference between the monitored frequency and the selected target frequency. Control proceeds to 716. At 716, the processor 204 calculates an absolute value of the determined difference calculated at 714. Control proceeds to 718. At 718, the processor 204 accesses the comparison module 220 and determines whether the absolute value of the determined difference calculated at 716 is greater than the frequency difference threshold selected at 710. If at 718, the condition is met, then control proceeds to 720 to generate and output and alert signal. Otherwise, control proceeds to 722 to continue monitoring the operating frequency of the micropump 210. At 720, the processor 204 generates and outputs an alert signal as previously described with respect to step 418 of process 400. At 722, the processor 204 accesses the counter module 218 and continues monitoring the actual operating frequency of the micropump 210. Control proceeds back to 714.

In various implementations, the processor 204 and counter module 218 may monitor an operating signal of the micropump 210. For example, the counter module 218 may monitor the output signal of the pump control module 216. In various implementations, as shown in FIG. 5, the operating signal of the micropump 210 may oscillate between an upper voltage limit and a lower voltage limit. As shown in FIG. 5, in some examples, the upper voltage limit may be about 16 volts, and the lower voltage limit may be about 0 volts. In various implementations, the processor 204 and/or the comparison module 220 may determine that liquid has entered the micropump 210 by comparing a number of cycles of the micropump 210 between the upper voltage limit and the lower voltage limit within a set time period. If the number of cycles exceeds a threshold number, then the comparison module 220 may determine that liquid has entered the micropump 210 and generate the alert signal. In various implementations, the processor 204 and/or the comparison module 220 may monitor the amount of time the micropump 210 spends at the upper voltage limit and/or the lower voltage limit within a normal running period. If the amount of time exceeds an upper threshold value and/or is less than a lower threshold value, then the comparison module 220 may determine that liquid has entered the micropump 210 and generate the alert signal.

In various implementations, the processor 204 and/or the comparison module 220 may monitor for a number of instances the upper voltage limit and/or the lower voltage limit is met within a time period. If the number of instances exceeds an upper threshold value and/or is less than a lower threshold value, then the comparison module 220 may determine that liquid has entered the micropump 210 and generate the alert signal. In various implementations, the processor 204 and/or the comparison module 220 may monitor for a rolling average of a number of instances the operating signal of the micropump 210 meets the upper voltage limit and/or the lower voltage limit, and monitor for deviations from a known normal condition. If the comparison module 220 determines that the rolling average deviates from the known normal condition, then the comparison module 220 may determine that liquid has entered the micropump 210 and generate the alert signal.

FIG. 8 is an isometric view of an assembled example of the dressing 104 with a liquid-detection sensor module 124 directly coupled to the cover 110 of the dressing 104 and fluidly coupled to the therapy unit 222. As shown in FIG. 8, the liquid-detection sensor module 124 may include a housing, such as a body 802. The body 802 may include a flanged portion 804 suitable for attachment to the cover 110. In various implementations, the flanged portion 804 may be directly coupled to a top surface 806 of the cover 110. For example, the bottom side of the flanged portion 804 may be attached to the top surface 806 of the cover 110 with an adhesive. In various implementations, the flanged portion 804 may carry a plurality of layers of adhesive. Each layer of the plurality of layers of adhesive may be separated from each other by a release liner positioned between each layer of the plurality of layers of adhesive. The release between each of the layers of adhesive may be configured to be removed to expose another of the layers of adhesive. In various implementations, fluid from the dressing 104 may exit the dressing 104 through an aperture (not shown) in the cover 110 underneath the liquid-detection sensor module 124, pass through the liquid-detection sensor module 124, and exit through an outlet 808 of the liquid-detection sensor module 124. As illustrated in FIG. 8, in some examples, the liquid-detection sensor module 124 may be fluidly coupled to the therapy unit 222 by a conduit, such as conduit 810. In various implementations, one end of the conduit 810 may be coupled to the outlet 808, while the opposite end of the conduit 810 may be fluidly coupled to the negative-pressure source 102 of the therapy unit 222.

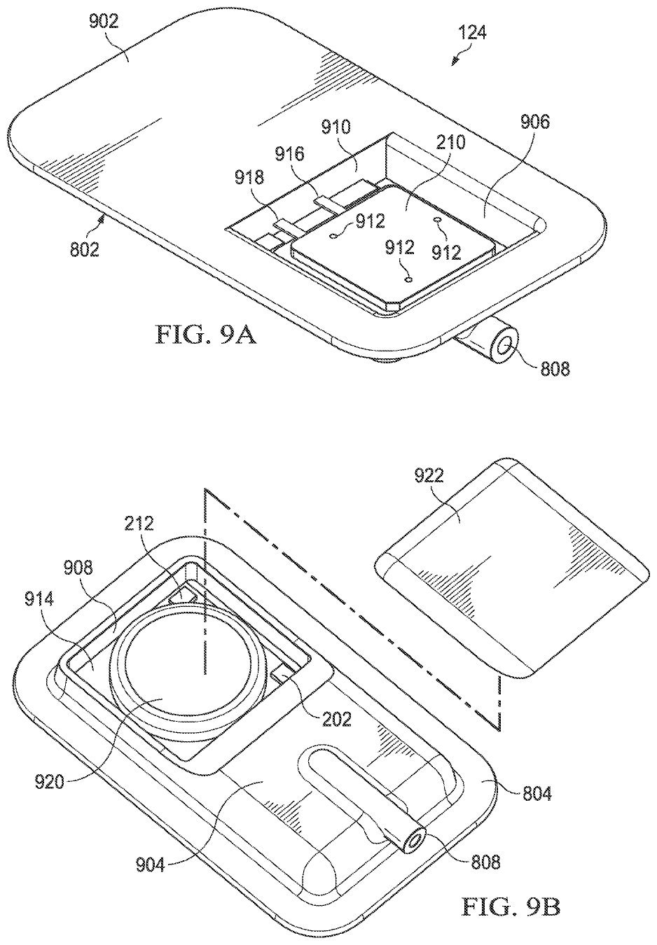

FIG. 9A is an isometric view of the bottom side of the liquid-detection sensor module 124 of FIG. 8. FIG. 9B is an isometric view of the top side of the liquid-detection sensor module 124 of FIG. 8. As shown in FIGS. 9A and 9B, the body 802 of the liquid-detection sensor module 124 may have a bottom surface 902 opposite a top surface 904. A first cavity, such as compartment 906 may be formed in the bottom surface 902. A second cavity, such as compartment 908, may be formed in the top surface 904. In various implementations, compartment 906 may be separated from compartment 908 by a walled portion, such as divider 910. The micropump 210 may be disposed within compartment 906, with air intakes 912 of the micropump 210 in fluid communication with the cavity formed by the compartment 906. The outlet (not shown) of the micropump 210 may be in fluid communication with the outlet 808 of the liquid-detection sensor module 124, but fluidly isolated from the cavity formed by the compartment 906. In various implementations, the bottom surface 902 of the body 802 may be coupled to the top surface 806 of the cover 110, and the compartment 906 may be in fluid communication with the interior of the dressing 104 through an aperture (not shown) formed in the cover 110.

In various implementations, a printed circuit board 914 may be disposed within the compartment 908. The microcontroller module 202, the pump driver 208, and the communications interface 212 may be provided on and operatively coupled to the printed circuit board 914. In various implementations, the microcontroller module 202, the pump driver 208, and the communications interface 212 may be provided as separate electronic components operatively coupled to the printed circuit board 914. In other implementations, one or more of the pump driver 208 and the communications interface 212 may be integrated with another electronic component, such as the microcontroller module 202. As shown in FIGS. 9A to 9B, without limitation, the pump driver 208 is presumed to be integrated with the microcontroller module 202 or the communications interface 212. The micropump 210 may be operatively coupled to the printed circuit board 914 through a first electrical connection 916 and a second electrical connection 918. In various implementations, the microcontroller module 202, the micropump 210, and the communications interface 212 may be directly or indirectly operatively coupled to each other through traces of the printed circuit board 914. A power source, such as battery 920, may be operatively coupled to the printed circuit board 914 and provide power to the microcontroller module 202, micropump 210, and communications interface 212. In various implementations, the user may access compartment 908 by removing a cover 922 disposed over compartment 908.

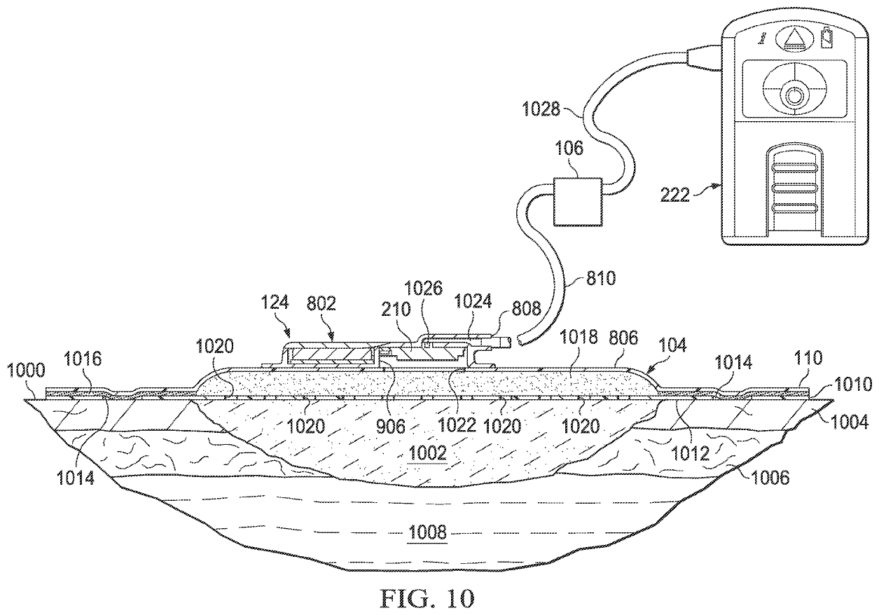

FIG. 10 is a cross-sectional view of the example dressing 104 and liquid-detection sensor module 124 of FIG. 8, taken at line 10-10, applied to an example tissue site 1000, and illustrating additional details associated with some examples of the therapy system 100 of FIG. 1. As illustrated in FIG. 10, the dressing 104 may be generally configured to be positioned adjacent to the tissue site 1000 and/or be in contact with a portion of the tissue site 1000, substantially all of the tissue site 1000, the tissue site 1000 in its entirety, or tissue around the tissue site 1000. In some examples, the tissue site 1000 may be or may include a defect or a targeted treatment site, such as a wound 1002. The wound 1002 may be partially or completely filled or covered by the dressing 104. In various implementations, the dressing 104 may take many forms, and may have many sizes, shapes, or thicknesses depending on a variety of factors, such as the type of treatment being implemented or the nature and size of the tissue site 1000. For example, the shape and size of the dressing 104 may be adapted to the contours of the wound 1002. In some examples, as shown in FIG. 10, the tissue site 1000 may include a wound 1002 which extends through the epidermis 1004, dermis 1006, and into subcutaneous tissue 1008.

As shown in FIG. 10, in some examples, the dressing 104 may include a tissue contact layer 1010. In various implementations, the tissue contact layer 1010 may be a soft, pliable material suitable for creating a fluid seal with the tissue site 1000. For example, the tissue contact layer 1010 may include a silicone gel, a soft silicone, a hydrocolloid, a hydrogel, a polyurethane gel, a polyolefin gel, hydrogenated styrenic copolymer gels, a foamed gel, a soft closed-cell foam such as a polyurethane or polyolefin coated with an adhesive, polyurethane, polyolefin, or hydrogenated styrenic copolymers. In various implementations, the tissue contact layer 1010 may have a thickness in a range of about 500 micrometers to about 1,000 micrometers. In various implementations, the tissue contact layer 1010 may have a stiffness in a range of between about 5 Shore OO to about 80 Shore OO. In various implementations, the tissue contact layer 1010 may be formed of hydrophobic or hydrophilic materials.

In various implementations, the tissue contact layer 1010 may be formed by coating a spaced material, such as woven, nonwoven, molded, or extruded mesh materials with a hydrophobic material. In various implementations, the hydrophobic materials for the coating may be a soft silicone. In various implementations, the spaced material may have sufficiently sized openings in the spaced material to permit an adhesive to extend through the openings.

In various implementations, the tissue contact layer 1010 may include an outer portion 1012 near the periphery of the tissue contact layer 1010, and a plurality of apertures 1014 formed through the outer portion. In various implementations, an adhesive layer 1016 may be disposed on at least a portion of the cover 110. In various implementations, the adhesive layer 1016 may include any of the materials previously described with respect to the attachment device of the cover 110. For example, the adhesive layer 1016 may be disposed on a portion of a side of the cover 110 facing the tissue contact layer 1010. In various implementations, the adhesive layer 1016 may cover at least a portion of the side of the cover 110 facing the tissue contact layer 1010 that is substantially coextensive with the outer portion 1012 of the tissue contact layer 1010. In various implementations, the adhesive layer 1016 may cover substantially an entirety of the side of the cover 110 facing the tissue contact layer 1010.

In various implementations, the tissue contact layer 1010 and the adhesive layer 1016 may work in conjunction with one another to seal and secure the dressing 104 to the tissue site 1000. For example, the tissue contact layer 1010 may be sufficiently tacky to hold the dressing 104 in position relative to the tissue site 1000 and the wound 1002, while simultaneously allowing the dressing 104 to be removed and/or repositioned without causing trauma to the wound 1002 or the epidermis 1004 surrounding the wound 1002. For example, as previously discussed, the tissue contact layer 1010 may include or be formed of a silicone material that may form sealing couplings at the surface of the epidermis 1004. In some embodiments, the bond strength or tackiness of the sealing couplings may have a peel adhesion or resistance to being peeled from a stainless steel material between about 0.5 N/25 mm to about 1.5 N/25 mm on stainless steel substrate at 25° C. at 50% relative humidity based on ASTM D3330. The tissue contact layer 1010 may achieve this bond strength after a contact time of less than 60 seconds. Tackiness may be considered a bond strength of an adhesive after a very low contact time between the adhesive and a substrate. In example embodiments, the tissue contact layer 1010 may have a thickness in a range of about 200 micrometers to about 1,000 micrometers. In the assembled state, the thickness of the tissue contact layer 1010 may create a gap between the adhesive layer 1016 and the epidermis 1004 through the apertures 1014 of the tissue contact layer 1010 such that the adhesive layer 15 is not in contact with the epidermis 1004.

As illustrated in FIG. 10, in use, the adhesive layer 1016 may be brought into contact with the epidermis 1004 by a force applied to a surface of the cover 110 facing away from the tissue site 1000. In use, if the assembled dressing 104 is in the desired location, the force may be applied to the cover 110 proximate the apertures 1014 to cause the adhesive layer 1016 to be pressed at least partially through the apertures 1014 and into contact with the epidermis 1004 to form bonding couplings. The bonding couplings may provide secure, releasable mechanical fixation of the dressing 104 to the epidermis 1004. In various implementations, the sealing couplings between the tissue contact layer 1010 and the epidermis 1004 may not be as mechanically strong as the bonding couplings between the adhesive layer 1016 and the epidermis 1004. In various implementations, the bonding couplings may securely anchor the dressing 104 to the epidermis 1004, inhibiting migration of the dressing 104.

In various implementations, the dressing 104 may include a core 1018 disposed between the cover 110 and the tissue contact layer 1010. In various implementations, the core 1018 may include any of the materials previously described with respect to the tissue interface 108. In various implementations, the core 1018 may include a hydrophilic material adapted to absorb fluids, such as liquids, from the wound 1002. In various implementations, core 1018 may include Luquafleece® material, Texsus FP2326, BASF 402C, Technical Absorbents 2317 available from Technical Absorbents, sodium polyacrylate super absorbers, cellulosics (carboxy methyl cellulose and salts such as sodium CMC), or alginates. In various implementations, the core 1018 may include a hydrophilic open-celled foam.

As illustrated in FIG. 10, the core 1018 may be disposed over a portion of the tissue contact layer 1010 having one or more apertures 1020 formed through the tissue contact layer 1010. In various implementations, an aperture 1022 may be formed through the cover 110. In various implementations, the apertures 1020 may be in fluid communication with the core 1018, and the aperture 1022 may also be in fluid communication with the core 1018. In various implementations, the apertures 1020 may be in fluid communication with the aperture 1022 through the core 1018. In various implementations and as illustrated in FIG. 10, the liquid-detection sensor module 124 may be coupled to the top surface 806 of the cover 110, and positioned such that the opening of the compartment 906 is positioned over the aperture 1022, and the interior of the compartment 906 is in fluid communication with the aperture 1022, the core 1018, and the apertures 1020.

In operation, negative pressure generated by the negative-pressure source 102, shown in FIG. 1 of the therapy unit 222 and/or micropump 210 may cause fluids, such as gases and liquids, to be removed from the wound 1002 through the apertures 1020 of the tissue contact layer 1010 and into the core 1018. From the core 1018, the fluids may travel through the aperture 1022 into the interior of compartment 906 of the body 802 of the micropump 210. Fluids may be pumped from the interior of the compartment 906 through the micropump 210 into a conduit 1024 formed in the body 802 of the liquid-detection sensor module 124. For example, fluids may enter the micropump 210 through the air intakes 912 and exit an outlet 1026 of the micropump 210 into the conduit 1024. Fluid may then exit the conduit 1024 through the outlet 808 of the liquid-detection sensor module 124 and travel through the conduit 810, the container 106, a conduit 1028, and to the negative-pressure source 102 of the therapy unit 222.

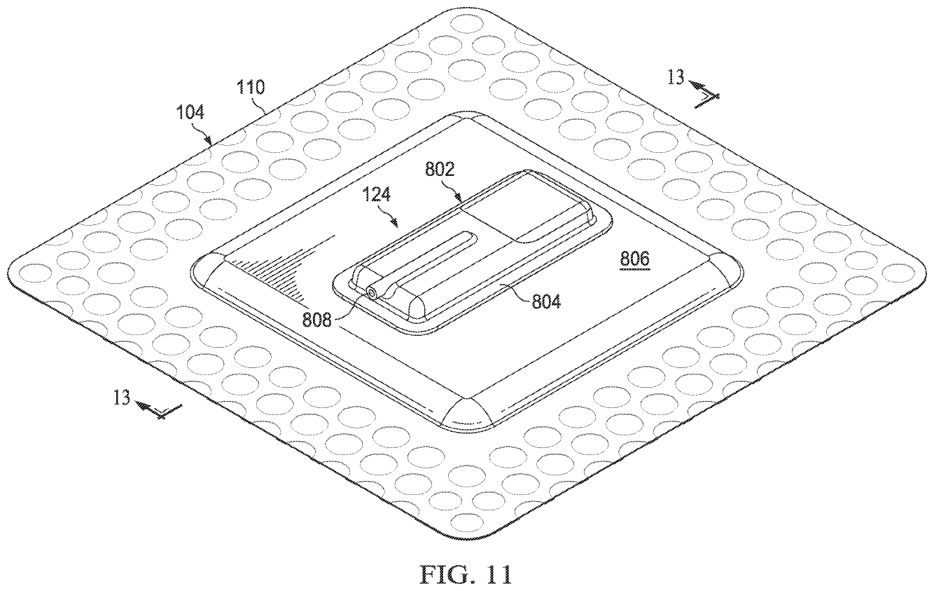

FIG. 11 is an isometric view of an assembled example of the dressing 104 with a liquid-detection sensor module 124 directly coupled to the cover 110 of the dressing 104 and fluidly coupled to the therapy unit 222. In various examples, the liquid-detection sensor module 124 of FIG. 11 may be substantially similar to the liquid-detection sensor module 124 of FIGS. 8-10, except that the liquid-detection sensor module 124 of FIG. 11 may include a second piezoelectric pump, such as the micropump 302, previously described in FIG. 3.

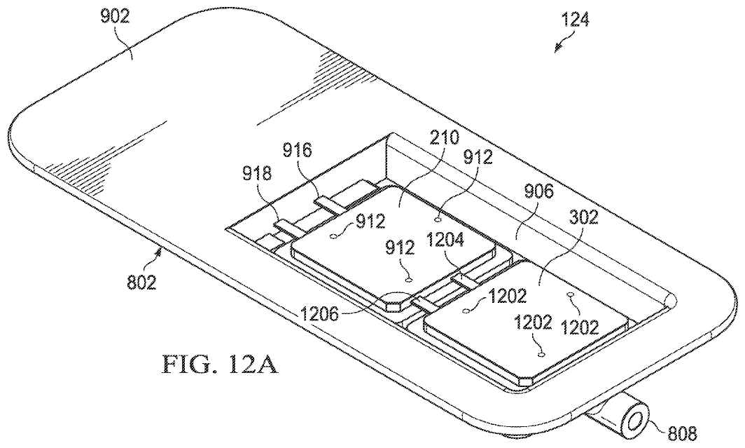

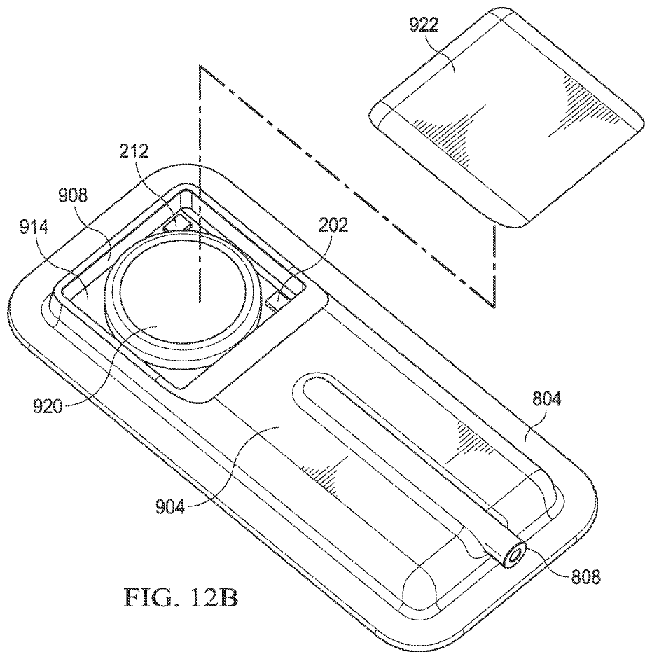

FIG. 12A is an isometric view of the bottom side of the liquid-detection sensor module 124 of FIG. 11. FIG. 12B is an isometric view of the top side of the liquid-detection sensor module 124 of FIG. 11. As illustrated in FIGS. 12A and 12B, the second micropump 302 may be disposed in the compartment 906 in addition to the first micropump 210. In various implementations, the micropump 302 may be positioned such that air intakes 1202 of the micropump 302 may be in fluid communication with the cavity formed by the compartment 906. In various implementations, the micropump 302 may be protected from liquid ingress with a gas-permeable but liquid-impermeable membrane, such as a hydrophobic filter. The outlet 1026, shown in FIG. 10, of the micropump 210 may be in fluid communication with the outlet 808 of the liquid-detection sensor module 124, but fluidly isolated from the cavity formed by the compartment 906. In various implementations, the micropump 302 may be operatively coupled to the printed circuit board 914 through a third electrical connection 1204 and a fourth electrical connection 1206. In various implementations, the micropump 302 may be operatively coupled to the microcontroller module 202 through the printed circuit board 914. In various implementations, the battery 920 may provide power to the micropump 302.

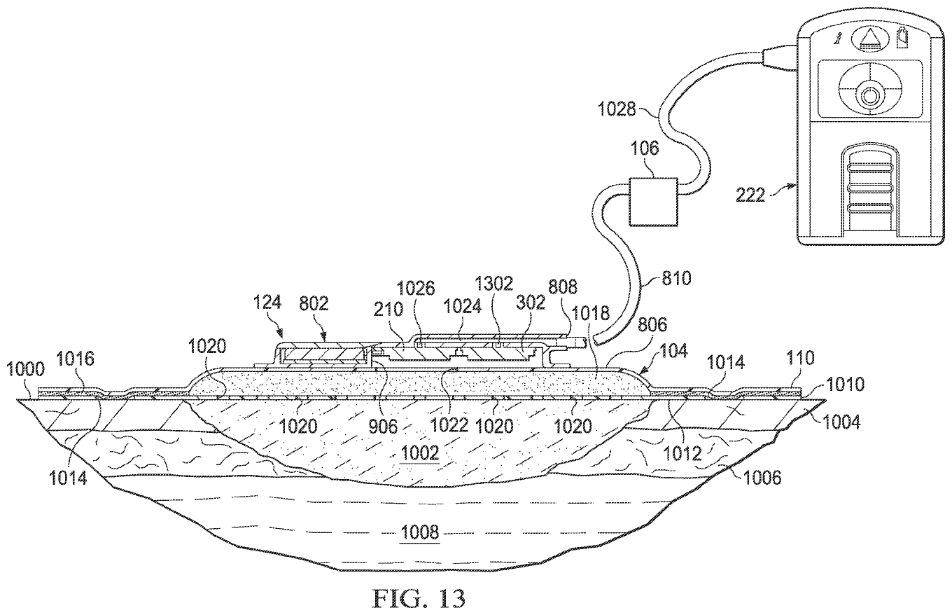

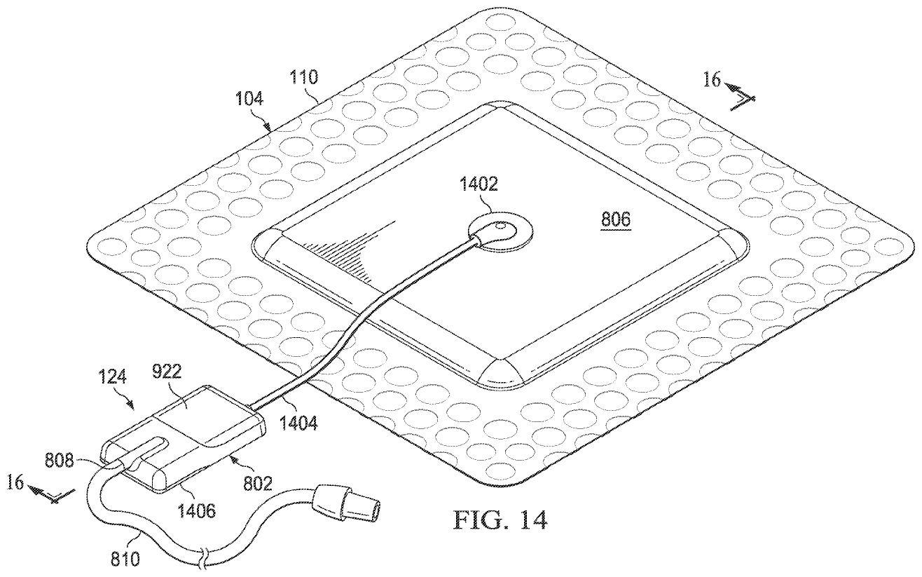

FIG. 13 is a cross-sectional view of the example dressing 104 and liquid-detection sensor module 124 of FIG. 11, taken at line 13-13, applied to an example tissue site 1000, and illustrating additional details associated with some examples of the therapy system 100 of FIG. 1. As previously described, in various implementations, the micropump 302 may provide negative pressure to the dressing 104 and/or the micropump 210. For example, the micropump 302 may remove fluid from the cavity formed by the compartment 906 through the air intakes 1202, shown in FIG. 12A, and exhaust the removed fluid through an outlet 1302 of the micropump 302. In various implementations, the micropump 302 may be isolated from the cavity formed by the compartment 906 by a hydrophobic filter. In various implementations, the hydrophobic filter may permit gases but not liquids to cross the membrane of the hydrophobic filter. In various implementations, each of the air intakes 1202 of the micropump 302 may be covered by one or more hydrophobic filters. In various implementations, the removed air may be exhausted from the outlet 1302 into the conduit 1024 of the liquid-detection sensor module 124. By removing fluid from the sealed interior space of the compartment 906, the micropump 302 may create negative pressure, which may be provided to the interior of the dressing 104 through the aperture 1022. In various implementations, if liquids have entered the micropump 210 through the air intakes 912, shown in FIG. 9A, which are in fluid communication with the cavity formed by the compartment 906, then the negative pressure created by the micropump 302 within the cavity formed by the compartment 906 may be effective in drawing fluid out from the inside of the micropump 210 through the air intakes 912.

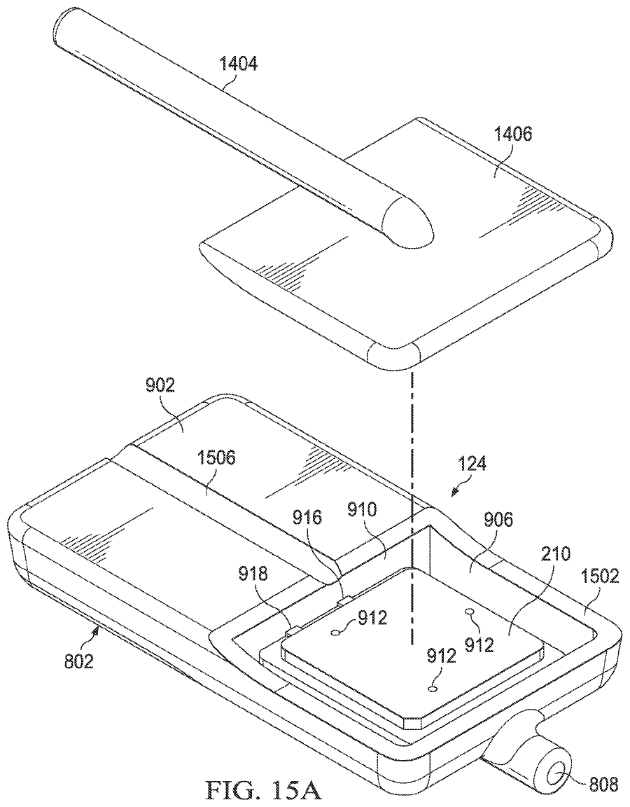

FIG. 14 is an isometric view of an assembled example of the dressing 104 with a liquid-detection sensor module 124 fluidly coupled to the dressing 104 through a conduit and a dressing interface. In various implementations, the liquid-detection sensor module 124 is not directly attached to the top surface 806 of the cover 110. For example, as illustrated in the example of FIG. 14, the liquid-detection sensor module 124 may be fluidly coupled to the dressing 104 through a dressing interface 1402 and a conduit 1404. In various implementations, the conduit 1404 may be fluidly coupled to the liquid-detection sensor module 124 using a sensor module connection interface 1406.

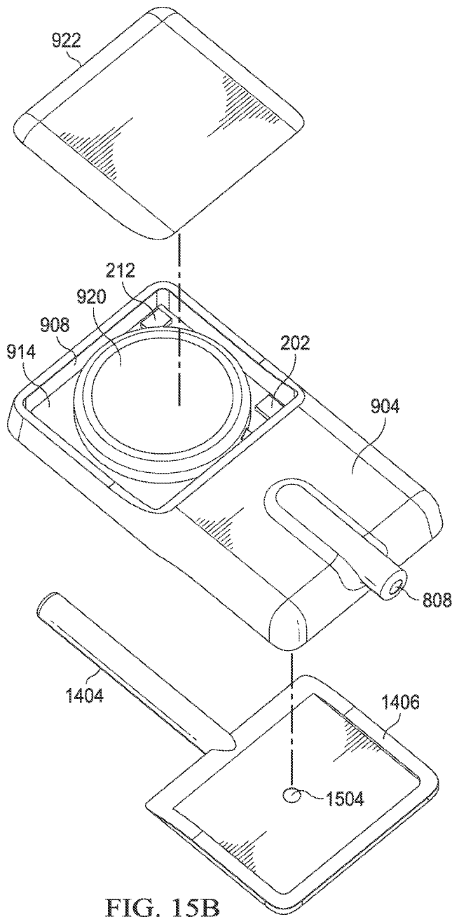

FIG. 15A is an isometric view of the bottom side of the liquid-detection sensor module 124 of FIG. 14. FIG. 15B is an isometric view of the top side of the liquid-detection sensor module 124 of FIG. 14. As shown in FIGS. 15A-15B, the conduit 1404 may be fluidly coupled to a sensor module connection interface 1406. The sensor module connection interface 1406 may be disposed over and cover the compartment 906. In various implementations, the sensor module connection interface 1406 seats against a perimeter wall 1502 of the compartment 906, fluidly isolating the cavity of the compartment 906 from the external ambient environment. In various implementations, the conduit 1404 may be in fluid communication with the cavity of the compartment 906 through a port 1504 formed through the sensor module connection interface 1406. In various implementations, a conduit channel 1506 for receiving and securing the conduit 1404 may be formed on the bottom surface 902 of the body 802.

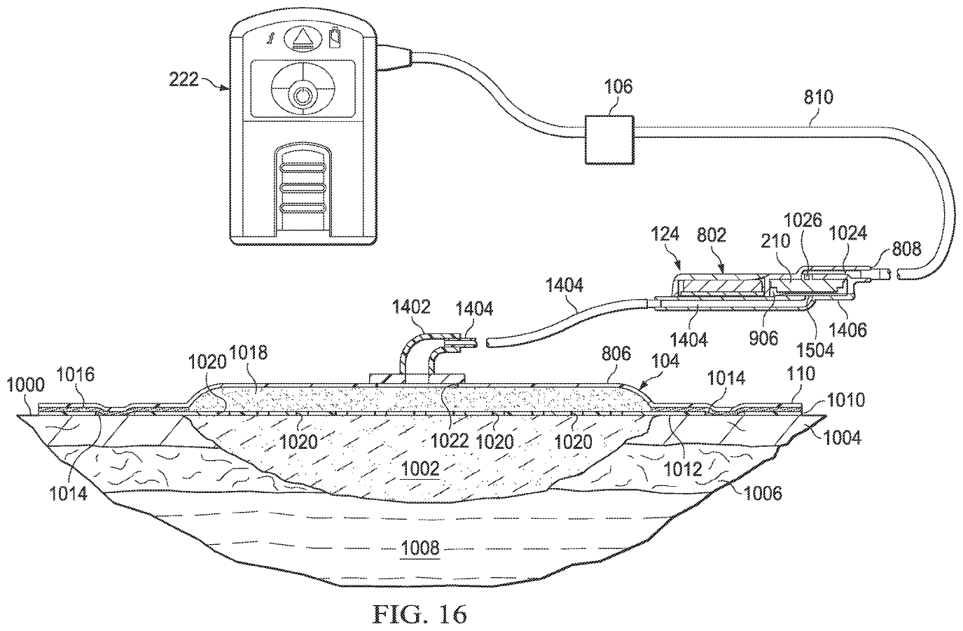

FIG. 16 is a cross-sectional view of the example dressing 104 and liquid-detection sensor module 124 of FIG. 14, taken at line 16-16, applied to an example tissue site 1000, and illustrating additional details associated with some examples of the therapy system 100 of FIG. 1. As illustrated in FIG. 16, in various implementations, the dressing interface 1402 may be positioned over the aperture 1022 of the cover 110 such that an interior cavity 1602 of the dressing interface 1402 is in fluid communication with the interior of the dressing 104 through the apertures 1022. In various implementations, the interior cavity 1602 of the dressing interface 1402 may be in fluid communication with the conduit 1404. In various implementations, negative pressure provided by the negative-pressure source 102 of the therapy unit 222 and/or micropump 210 may cause fluids to travel from the core 1018 of the dressing 104, through the aperture 1022, and into the interior cavity 1602 of the dressing interface 1402. From the interior cavity 1602, fluids enter the conduit 1404 and travel through the port 1504 and into the interior space formed between the compartment 906 of the liquid-detection sensor module 124 and the sensor module connection interface 1406. From the interior space of the compartment 906, fluids may enter the air intakes 912, shown in FIG. 9A, of the micropump 210.

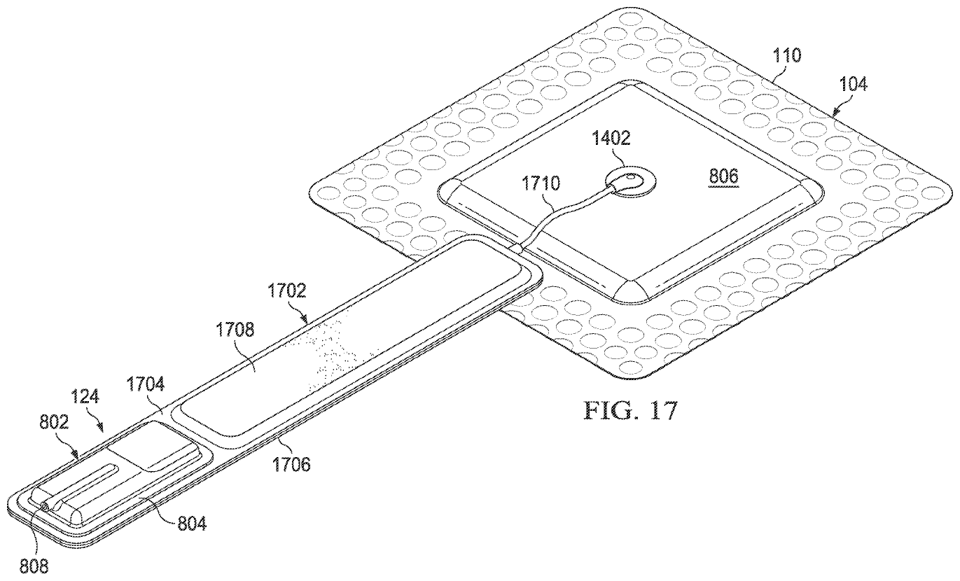

FIG. 17 is an isometric view of an assembled example of the dressing 104 with a liquid-detection sensor module 124 fluidly coupled to the dressing 104 through a conduit and a bridge. In various implementations, the therapy system 100 may include a bridge 1702. The bridge 1702 may be formed from a first layer 1704 coupled to a second layer 1706 at the periphery of the first layer 1704 and the second layer 1706 to define an interior space between the first layer 1704 and the second layer 1706. In various implementations, the first layer 1704 and/or the second layer 1706 may be formed from any of the materials previously described with respect to cover 110. Further, the first layer 1704 and the second layer 1706 may be replaced with a single layer folded upon itself to define the interior space. In various implementations, an absorbent and/or manifolding core 1708 may be disposed in the interior space of the bridge 1702 between the first layer 1704 and the second layer 1706. In FIG. 17, the core 1708 is visible through the transparent first layer 1704. In various implementations, the interior space of the bridge 1702 may be fluidly coupled to the interior space of the dressing 104 through the dressing interface 1402 and a conduit 1710. For example, fluid may travel from the core 1018 of the dressing 104 to the core 1708 of the bridge 1702 via the dressing interface 1402 and the conduit 1710. In various implementations, the cavity formed by compartment 906 of the liquid-detection sensor module 124 may be positioned over an aperture (not shown) formed through the first layer 1704 of the bridge 1702. In this way, the micropump 210 may be in fluid communication with the core 1708 of the bridge 1702.

FIG. 18 is a cross-sectional view of a container 106 with a liquid-detection sensor module 124 disposed in an interior space of the container 106. In various implementations, container 106 may include a plurality of walls. For example, the container 106 may include one or more walls, such as sidewalls 1802, a top wall 1804, and a bottom wall 1806. The sidewalls 1802, top wall 1804, and bottom wall 1806 may define the interior space 1808 of the container 106. In various implementations, the liquid-detection sensor module 124 may be attached to a wall of the container 106. For example, as illustrated in FIG. 18, the liquid-detection sensor module 124 may be attached to the top wall 1804. In various implementations, the liquid-detection sensor module 124 may be attached to a sidewall 1802 at a position at which it is desirable to detect a presence of liquids. In examples where the liquid-detection sensor module 124 is attached one of the walls of the container 106, a port 1810 may be formed through the wall of the container 106 to which the liquid-detection sensor module 124 is attached. The outlet 1026 of the micropump 210 may be in fluid communication with an external environment via the outlet 1026. In various implementations, compartment 906 of the liquid-detection sensor module 124 may be in fluid communication with the interior space 1808.

In operation, the micropump 210 may draw fluid from the interior space 1808 of the container 106 into compartment 906 of the liquid-detection sensor module 124, and from compartment 906 into the body of the micropump 210 through one or more of the air intakes 912 of the micropump 210. The micropump 210 may then exhaust gas through the outlet 1026 of the micropump 210 into the external environment via the port 1810 formed through one of the walls of the container 106. When a liquid level rises in the interior space 1808 to reach the micropump 210 such that liquid is drawn into the air intakes 212, the liquid exposure of the micropump 210 may cause a deviation in the operating frequency of the micropump 210, which could signal an alarm as previously described. In such a condition, the micropump 210 will not allow liquid to exit the outlet 1026 of the micropump 210.

The systems, apparatuses, and methods described herein may provide significant advantages. For example, the liquid-detection sensor module 124 may detect the presence of liquid in any of the components and/or fluid pathways of the therapy system 100. In various implementations, the liquid-detection sensor module 124 may use any combination of processes 400 and/or 700 to detect when liquid has entered the liquid-detection sensor module 124. In some examples, the liquid-detection sensor module 124 may use any combination of processes 400 and/or 700 to detect when liquid has entered the micropump 210, which may be disposed within the compartment 906. For example, the liquid-detection sensor module 124 may use any combination of processes 400 and/or 700 to detect when liquid has entered the micropump 210 through air intakes 912. In various implementations, when the liquid-detection sensor module 124 is fluidly coupled to the dressing 104, the liquid-detection sensor module 124 detecting liquid may be indicative of the core 1018 of the dressing 104 being saturated with liquids from the wound 1002. In various implementations, when the liquid-detection sensor module 124 is fluidly coupled to the bridge 1702, the liquid-detection sensor module 124 detecting liquids may be indicative of the core 1708 of the bridge 1702 being saturated with liquids from the wound 1002. In various implementations, when the liquid-detection sensor module 124 is fluidly coupled to the interior of the container 106, the liquid-detection sensor module 124 detecting liquids may be indicative of the container 106 being full, or having reached the location of the liquid-detection sensor module 124.

After the liquid-detection sensor module 124 detects the presence of liquid, an alert signal may be sent to the therapy unit 222 and/or the user device 224, and the therapy unit 222 and/or the user device 224 may alert the user that the relevant component of the therapy system 100 is saturated with liquid and/or shut down the negative-pressure source 102 of the therapy unit 222. By alerting the user or automatically shutting down the negative-pressure source 102, the therapy unit 222 may be protected from damage caused by liquids ingress.

Additionally, due to the relatively low cost of the liquid-detection sensor module 124, these units may be disposed of with the dressing 104. Furthermore, because liquids are not transported across the micropump 210 or the micropump 302, the liquid-detection sensor module 124 also acts as a liquid filter, preventing liquids from being transported across the liquid-detection sensor module 124.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may also be combined or eliminated in various configurations for purposes of sale, manufacture, assembly, or use. For example, in some configurations the dressing 104, the container 106, or both may be eliminated or separated from other components for manufacture or sale. In other example configurations, the controller 112 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A system for treating a tissue site with negative pressure, comprising:
a dressing configured to be positioned at the tissue site;
a negative-pressure source configured to supply the negative pressure to the dressing through a fluid pathway;
a micropump configured to be positioned in fluid communication with the fluid pathway; and
a controller associated with the micropump and configured to generate an alert signal if a deviation condition is met in an actual operating frequency of the micropump.

2. The system of claim 1, wherein the micropump is configured to be positioned in fluid communication between the dressing and the negative-pressure source in series such that the negative pressure from the negative-pressure source is supplied to the dressing through the micropump.

3. The system of claim 1, wherein the negative-pressure source is configured to supply the negative pressure to the dressing entirely through the micropump.

4. (canceled)

5. The system of claim 1, wherein an intake of the micropump is configured to be directly exposed to a liquid in the fluid pathway.

6. The system of claim 1, wherein the micropump is a first micropump, and wherein the system further comprises a second micropump in fluid communication with the first micropump and the fluid pathway, wherein an intake of the first micropump is configured to be directly exposed to a liquid in the fluid pathway, and wherein an intake of the second micropump is configured to be isolated from the liquid by a liquid-impermeable membrane.

7. (canceled)

8. The system of claim 1, further comprising a housing including a first cavity fluidly isolated from a second cavity by a walled portion, wherein the micropump is positioned in the first cavity, and wherein the controller is positioned in the second cavity.

9. The system of claim 8, wherein the second cavity further comprises a power source and a wireless communications interface.

10. The system of claim 8, wherein the first cavity is configured to be positioned in fluid communication with the fluid pathway.

11. The system of claim 8, wherein the housing includes a flanged portion surrounding the first cavity.

12. The system of claim 11, wherein the flanged portion carries at least one layer of adhesive configured to couple the first cavity in fluid communication with the fluid pathway, wherein the at least one layer of adhesive is a plurality of layers of adhesive separated from each other by a release liner positioned between each layer of the plurality of layers of adhesive, and wherein the release liner between each of the layers of adhesive is configured to be removed to expose another of the layers of adhesive.

13. (canceled)

14. The system of claim 1, wherein the micropump is configured to be fluidly coupled to the fluid pathway through one or more of a cover of the dressing, an in-line conduit between the dressing and the negative-pressure source, a fluid canister between the negative-pressure source and the dressing, and a bridge between the negative-pressure source and the dressing.

15. The system of claim 1, wherein the controller is configured to determine the deviation condition by:
monitoring the actual operating frequency of the micropump;
comparing the actual operating frequency to a target frequency; and
determining the deviation condition in response to the actual operating frequency deviating from the target frequency.

16. (canceled)

17. (canceled)

18. (canceled)

19. The system of claim 1, wherein the controller is configured to determine the deviation condition by:
monitoring the actual operating frequency of the micropump;
determining a difference between the actual operating frequency and a target frequency of the micropump;
calculating an absolute value of the difference;
determining whether the absolute value of the difference exceeds a frequency difference threshold; and
determining the deviation condition in response to the absolute value of the difference exceeding the frequency difference threshold.

20. A system for providing negative-pressure wound therapy, comprising:
a dressing configured to be fluidly coupled to a negative-pressure source; and
a liquid-detection sensor module configured to be fluidly coupled to a fluid pathway between the dressing and the negative-pressure source, comprising:
a controller, and
a micropump operatively associated with the controller;
wherein the controller is configured to:
monitor an actual operating frequency of the micropump,
compare the actual operating frequency to a target frequency,
determine whether a deviation condition is met when the actual operating frequency deviates from the target frequency, and
generate an alert signal if the deviation condition is met.

21. (canceled)

22. (canceled)

23. (canceled)

24. The system of claim 20, wherein the controller is further configured to:
determine a difference between the actual operating frequency and the target frequency;
calculate an absolute value of the difference;
determine whether the absolute value of the difference exceeds a frequency difference threshold; and
determine that the deviation condition is met if the absolute value of the difference exceeds the frequency difference threshold.

25. The system of claim 20, wherein the controller is further configured to send the alert signal to one or both of a user device and a therapy unit.

26. The system of claim 25, wherein the therapy unit is configured to:
receive the alert signal; and
shut off a negative-pressure source of the therapy unit in response to receiving the alert signal.

27. The system of claim 25, wherein the therapy unit is configured to:
receive the alert signal; and
generate a user notification in response to receiving the alert signal.

28. The system of claim 20, further comprising:
a user device operatively coupled to the controller;
wherein the controller is further configured to:
send the alert signal to the user device;
wherein the user device is configured to:
receive the alert signal, and
generate a user notification in response to receiving the alert signal.

29. A non-transitory computer-readable medium comprising executable instructions for generating an alert signal indicative of liquid saturation in a therapy system, wherein the executable instructions configure a controller to:
monitor an actual operating frequency of a piezoelectric micropump;
compare the actual operating frequency to a target frequency;
determine whether a deviation condition is met when the actual operating frequency deviates from the target frequency; and
generate an alert signal if the deviation condition is met.

30. (canceled)
